(19) United States
(12) Patent Application Publication
(10) Pub. No.: US 2025/0369051 A1
(43) Pub. Date: Dec. 4, 2025

(54) NOVEL USE OF CTDNA TO IDENTIFY LOCALLY ADVANCED AND METASTATIC UPPER TRACT UROTHELIAL CARCINOMA

(71) Applicant: H. LEE MOFFITT CANCER CENTER AND RESEARCH INSTITUTE, INC., Tampa, FL (US)

(72) Inventors: Roger LI, Tampa, FL (US); Liang WANG, Tampa, FL (US)

(21) Appl. No.: 18/864,198

(22) PCT Filed: May 10, 2023
(86) PCT No.: PCT/US2023/021703

§ 371 Date: Nov. 8, 2024

Related U.S. Application Data
(60) Provisional application No. 63/340,140, filed on May 10, 2022.

(51) Int. Cl. C12Q 1/6886

(52) U.S. Cl. CPC C12Q 1/6886; C12Q 2600/112; C12Q 2600/118; C12Q 2600/156; G01N 2800/52

(57) ABSTRACT

Disclosed are compositions and methods for detecting, prognosing, grading, and treating a cancer such as, for example a bladder cancer including, but not limited to upper tract urothelial carcinoma (UTUC) using circulating tumor DNA (ctDNA).

### I. CROSS REFERENCE TO RELATED APPLICATIONS

This application claims the benefit of U.S. Provisional Application No. 63/340,140, filed on May 10, 2022, which is incorporated herein by reference in its entirety.

### II. BACKGROUND

Upper tract urothelial carcinoma (UTUC) is an aggressive cancer for which use of neoadjuvant chemotherapy (NAC) is limited by suboptimal clinical staging prior to nephroureterectomy. Detection of circulating tumor DNA (ctDNA) associated with locally advanced and nodally metastatic urothelial carcinoma of the bladder may help identify UTUC patients who would benefit from NAC. Optimal patient selection for neoadjuvant chemotherapy prior to surgical extirpation is limited by the inaccuracy of contemporary clinical staging methods in high-risk upper tract urothelial carcinoma (UTUC). What are needed are new methods to detect cancers and assess cancer risk so appropriate treatments can be applied to patients and thereby increase cancer survivability.

### III. SUMMARY

Disclosed are methods and compositions related detecting, prognosing, grading, and treating a cancer such as, for example a bladder using circulating tumor DNA (ctDNA).

In one aspect, disclosed herein are methods of detecting the presence of a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) in a subject comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); and b) assaying circulating tumor DNA (ctDNA) in the tissue sample using next generation sequencing (NGS) or whole genome sequencing (WGS) to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGTIA1, VHL, XPC, and XRCC1; wherein the presence of two or more genes indicates the presence of a cancer. In one aspect, the gene alteration comprises a TP53, TERT, MYC, FGFR3, CDKN2A, ATM, or ARIDIA alteration.

Also disclosed herein are methods of detecting the presence of a cancer and/or metastasis of any preceding aspect, further comprising extracting DNA from the tissue sample.

In one aspect, disclosed herein are methods of detecting the presence of a cancer and/or metastasis of any preceding aspect, further comprising measuring plasma copy number burden (CNB); wherein a CNB of >6.5 indicates the presence of a cancer.

Also disclosed herein are methods of detecting the presence of a cancer and/or metastasis of any preceding aspect, further comprising administering to the subject an anti-cancer treatment (such as, for example a cisplatin-based neoadjuvant chemotherapy or nephroureterectomy (RNU)) when a cancer is detected.

In one aspect, disclosed herein are methods of predicting survival (including overall survival and/or progression free survival) in a subject treated for a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC), the method comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); and b) assaying circulating tumor DNA (ctDNA) in the tissue sample using next generation sequencing to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGT1A1, VHL, XPC, and XRCC1; wherein the presence of two or more genes indicates an aggressive cancer and low chance survival. In one aspect, the gene alteration comprises a TP53, TERT, MYC, FGFR3, CDKN2A, ATM, or ARIDIA alteration. In some aspect, the method is performed after nephroureterectomy (RNU).

Also disclosed herein are methods of predicting survival (including overall survival and/or progression free survival) of any preceding aspect, further comprising extracting DNA from the tissue sample.

In one aspect, disclosed herein are methods of predicting survival (including overall survival and/or progression free survival) of any preceding aspect, further comprising measuring plasma copy number burden (CNB); wherein a CNB of >6.5 indicates the presence of a cancer.

Also disclosed herein are methods of predicting survival (including overall survival and/or progression free survival) of any preceding aspect, further comprising administering to the subject an anti-cancer treatment (such as, for example a cisplatin-based neoadjuvant chemotherapy) when cancer survival is low.

In one aspect, disclosed herein are methods of staging the severity of a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) in a subject comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); and b) assaying circulating tumor DNA (ctDNA) in the tissue sample using next generation sequencing to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGT1A1, VHL, XPC, and XRCC1; wherein the presence of two or more genes indicates the presence of an aggressive cancer. In one aspect, the gene alteration comprises a TP53, TERT, MYC, FGFR3, CDKN2A, ATM, or ARIDIA alteration.

Also disclosed herein are methods of staging cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) of any preceding aspect, further comprising extracting DNA from the tissue sample.

In one aspect, disclosed herein are methods of staging cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) of any preceding aspect, further comprising measuring plasma copy number burden (CNB); wherein a CNB of >6.5 indicates the presence of a cancer.

Also disclosed herein are methods of staging cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) of any preceding aspect, further comprising administering to the subject an anti-cancer treatment (such as, for example, a cisplatin-based neoadjuvant chemotherapy or nephroureterectomy (RNU)) when an aggressive cancer is detected.

In one aspect, disclosed herein are methods of treating, inhibiting, reducing, decreasing, ameliorating, and/or preventing a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) in a subject comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); b) assaying circulating tumor DNA (ctDNA) in the tissue sample using next generation sequencing (NGS) or whole genome sequencing (WGS) to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGT1A1, VHL, XPC, and XRCC1; wherein the presence of two or more genes indicates the presence of a cancer; and c) administering to the subject an anti-cancer treatment (such as, for example a cisplatin-based neoadjuvant chemotherapy or nephroureterectomy (RNU)) when a cancer is detected. In one aspect, the gene alteration comprises a TP53, TERT, MYC, FGFR3, CDKN2A, ATM, or ARIDIA alteration.

Also disclosed herein are methods of treating, inhibiting, reducing, decreasing, ameliorating, and/or preventing a cancer and/or metastasis of any preceding aspect, further comprising extracting DNA from the tissue sample.

In one aspect, disclosed herein are methods of treating, inhibiting, reducing, decreasing, ameliorating, and/or preventing a cancer and/or metastasis of any preceding aspect, further comprising measuring plasma copy number burden (CNB); wherein a CNB of >6.5 indicates the presence of a cancer.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows mutation allelic frequency (MAF) comparison of detected mutations in matched UTUC tumor tissue and plasma-derived ctDNA

FIG. 2 shows detectable UTUC tumor tissue and preoperative plasma-derived ctDNA mutational profiles for individual patients by stage.

FIGS. 3A and 3B show a summary of molecular alteration profiling, histopathologic, and clinical characteristics for 16 NMI and 14 MI/NOC UTUC patients with paired tumor tissue and preoperative plasma cfDNA profiles ordered by advancing pathologic T stage. Each column represents one patient. Upper filled triangles represent plasma mutations and lower open triangles represent tumor tissue mutations.

FIG. 3C shows a Venn diagram showing overall mutational concordance between tumor tissue and plasma cfDNA.

FIGS. 4A, 4B, 4C, and 4D show numbers of molecular alterations and their frequencies found in the tumor tissue (4A and 4B) and plasma cfDNA (4C and 4D). There was a significantly higher number of alterations observed in the plasma of MI/NOC vs. NMI patients (3.4 vs. 0.5, p<0.0001), but not in the tumor tissue (7.0 vs 8.3, p=0.52). No gene was significantly more frequently altered in MI/NOC compared to NMI tumor tissue. However, alterations were more frequently found in TP53, TERT, and ARIDIA in the plasma from MI/NOC patients (*). Tissue molecular alterations found in 3 or more patients and plasma alterations in 1 or more are shown.

FIGS. 5A and 5B show an event chart showing patients with 5A) NMI and 5B) MI/NOC UTUC. Preoperative ctDNA positivity (</=2 plasma variants detected) is shown as open and filled triangles at time of surgery. Urothelial recurrences and progression to metastases are shown during follow up along with adjuvant chemotherapy treatment.

FIGS. 6A and 6B show the prognostic value of ctDNA detection. FIG. 6A shows progression-free and 6B overall survival were significantly prolonged for those patients who were ctDNA positive at the time of extirpative surgery. One-year PFS was 69% for patients with positive preoperative ctDNA compared to 100% for patients with negative ctDNA (p<0.001). Similarly, one-year OS was 56% compared to 100% (p=0.016) at a median 12.7 months of follow up.

FIG. 7 shows the genes included in the PredicineCARE™ assay. The panel interrogates 152 genes, including 103 genes with complete exonic coverage and 49 genes with select exonic coverage (indicated with *). PredicineCARE™ also includes parallel low-pass WGS sequencing used to generate CNB Score and analysis of large-scale chromosomal amplifications and deletions.

FIG. 8 shows UTUC tumor tissue-based TMB scores for patients. The highest scoring patient with TMB>20 mut./Mb can be classified as hypermutated.

FIG. 9 shows the receiver-operating curve for prediction of MI/NOC UTUC from preoperative plasma cfDNA variant count (including SNVs, indels, and CNV).

FIG. 10 shows SNV counts, CNV counts, and CNB scores from tumor tissue for patients from this study classified according to mutational subtypes defined by Fujii et al. 2021. Patients with the TP53+ mutational subtype have a significantly higher number of gene-level CNVs (1.9 vs. 3.7, p=0.03) and a marginally higher number of SNV/indels (3.8 vs. 5.6, p=0.13) and genome-wide copy-number burden (9.1 vs. 9.8, p=0.09). The hypermutated subtype sample is also positive for TP53 mutation.

FIG. 11A shows the CNB score of tumor tissues and preoperative plasma cfDNA for NMI and MI/NOC patients.

FIG. 11B shows the Plasma CNB score vs. number of variants observed for each patient. Setting a threshold of plasma CNB Score >6.5 (horizontal dotted line) to confirm MI/NOC disease for patients with >2 observed plasma variants (vertical dotted line) adds one additional true positive call (upper left quadrant) without any reduction in specificity. Overall sensitivity of this stepwise method is 79% at 94% specificity.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. KITS

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### C. METHOD OF TREATING CANCER

Upper tract urothelial carcinoma (UTUC) is an aggressive disease with up to 70% incidence of high-grade histology and 60% muscle-invasive staging at the time of radical nephroureterectomy (RNU). Patients with muscle-invasive UTUC (≥pT2) have a poor prognosis, with 5-year cancer-specific mortality rates ranging between 21-59%. Fortunately, there is emerging evidence that cisplatin-based neoadjuvant chemotherapy (NAC) can be safely delivered to achieve pathologic down-staging and improved survival. However, a major challenge preventing optimal patient selection for NAC rests with the difficulty of accurate clinical staging due to UTUC's cloistered anatomical location. Tumor biopsies by ureteroscopy under-stage UTUC up to 46% of the time. Efforts to improve clinical risk stratification using nomograms incorporating ureteroscopic findings, histologic features and cross-sectional imaging yielded only incremental gains. Moreover, clinical under-staging causes missed opportunities for systemic therapy, as those patients who develop renal insufficiency after surgery can no longer qualify for chemotherapy. Given the high stakes for accurate preoperative risk stratification, predictive biomarkers for invasive UTUC are critically needed.

The detection of circulating tumor DNA (ctDNA), that is, plasma cell-free DNA with tumor-specific alterations, is increasingly adopted for numerous clinical applications including cancer diagnosis, assessment of treatment response, and detection of residual disease and/or recurrence ctDNA can be detected in up to 35% of patients with localized urothelial carcinoma of the bladder and 83% with metastatic urothelial cancer. Saliently, higher levels of ctDNA have been shown to correlate with disease burden and portend worse outcomes. It was demonstrated higher levels of ctDNA in patients with muscle invasive bladder cancer than those with recurrent non-muscle invasive disease. Based on these findings, we hypothesized that the detection of plasma ctDNA can be used to refine clinical staging in high-risk UTUC patients undergoing extirpative surgery. In this study, we demonstrate the feasibility of preoperative ctDNA collection and correlate its accuracy in the prediction of muscle-invasive and non-organ confined UTUC (MI/NOC UTUC).

In one aspect, disclosed herein are methods of detecting the presence of a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) in a subject comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); and b) assaying circulating tumor DNA (ctDNA) in the tissue sample using next generation sequencing (NGS) or whole genome sequencing (WGS) to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGTIA1, VHL, XPC, and XRCC1; wherein the presence of two or more genes/gene alterations indicates the presence of a cancer. In one aspect, the gene alteration comprises a TP53, TERT, MYC, FGFR3, CDKN2A, ATM, or ARIDIA alteration.

Also disclosed herein are methods of detecting the presence of a cancer and/or metastasis of any preceding aspect, further comprising extracting DNA from the tissue sample.

In one aspect, disclosed herein are methods of detecting the presence of a cancer and/or metastasis of any preceding aspect, further comprising measuring plasma copy number burden (CNB); wherein a CNB of >6.5 indicates the presence of a cancer.

Also disclosed herein are methods of detecting the presence of a cancer and/or metastasis of any preceding aspect, further comprising administering to the subject an anti-cancer treatment (such as, for example a cisplatin-based neoadjuvant chemotherapy or nephroureterectomy (RNU)) when a cancer is detected.

It is understood and herein contemplated that the disclosed methods can be used to stage (i.e,. grade) a cancer and/or distinguish aggressive cancers from non-aggressive/less aggressive cancers. Accordingly, in one aspect, disclosed herein are methods of staging the severity of a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) in a subject comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); and b) assaying circulating tumor DNA (ctDNA) in the tissue sample using next generation sequencing to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCD1LG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGT1A1, VHL, XPC, and XRCC1; wherein the presence of two or more genes/gene alterations indicates the presence of an aggressive cancer. In one aspect, the gene alteration comprises a TP53, TERT, MYC, FGFR3, CDKN2A, ATM, or ARIDIA alteration.

Also disclosed herein are methods of staging cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) of any preceding aspect, further comprising extracting DNA from the tissue sample.

In one aspect, disclosed herein are methods of staging cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) of any preceding aspect, further comprising measuring plasma copy number burden (CNB); wherein a CNB of >6.5 indicates the presence of a cancer.

Also disclosed herein are methods of staging cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) of any preceding aspect, further comprising administering to the subject an anti-cancer treatment (such as, for example, a cisplatin-based neoadjuvant chemotherapy or nephroureterectomy (RNU)) when an aggressive cancer is detected.

Because the disclosed methods can assess the aggressiveness of a cancer or stage a cancer, the same methods can be used to determine a patients survivability or progression free survival, with the detection of a cancer and/or an aggressive cancer being an indicator or poor survivability or low chance or progression free survival and absence of a detected cancer indicating high progression free survival. Thus, in one aspect, disclosed herein are methods of predicting survival (including overall survival and/or progression free survival) in a subject treated for a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC), the method comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); and b) assaying circulating tumor DNA (ctDNA) in the tissue sample using next generation sequencing to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGT1A1, VHL, XPC, and XRCC1; wherein the presence of two or more genes/gene alterations indicates an aggressive cancer and/or low chance survival and less than two gene indicates a high chance of progression free survival. In one aspect, the gene alteration comprises a TP53, TERT, MYC, FGFR3, CDKN2A, ATM, or ARIDIA alteration. In some aspect, the method is performed after nephroureterectomy (RNU).

Also disclosed herein are methods of predicting survival (including overall survival and/or progression free survival) of any preceding aspect, further comprising extracting DNA from the tissue sample.

In one aspect, disclosed herein are methods of predicting survival (including overall survival and/or progression free survival) of any preceding aspect, further comprising measuring plasma copy number burden (CNB); wherein a CNB of >6.5 indicates the presence of a cancer.

Also disclosed herein are methods of predicting survival (including overall survival and/or progression free survival) of any preceding aspect, further comprising administering to the subject an anti-cancer treatment (such as, for example a cisplatin-based neoadjuvant chemotherapy) when cancer survival is low.

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer (including, but not limited to upper tract urothelial carcinoma (UTUC) such as muscle-invasive (MI)/non-organ confined (NOC) (MI/NOC) UTUC or non-muscle invasive (NMI) UTUC); urinary tract cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

In one aspect, disclosed herein are methods of treating, inhibiting, reducing, decreasing, ameliorating, and/or preventing a cancer and/or metastasis (such as, for example, a bladder or urinary tract cancer including, but not limited to upper tract urothelial carcinoma (UTUC) such as muscle-invasive (MI)/non-organ confined (NOC) (MI/NOC) UTUC or non-muscle invasive (NMI) UTUC) in a subject comprising a) obtaining a tissue sample from the subject (such as, for example a liquid biopsy including, but not limited to a liquid biopsy comprising whole blood, peripheral blood, plasma, serum, saliva, sputum, cerebral spinal fluid, urine, or lymph); b) assaying circulating tumor DNA (ctDNA) in the tissue sample using next generation sequencing (NGS) or whole genome sequencing (WGS) to detect the presence of alternations (such as a somatic mutation) in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGTIA1, VHL, XPC, and XRCC1; wherein the presence of two or more genes/gene alterations indicates the presence of a cancer; and c) administering to the subject an anti-cancer treatment (such as, for example a cisplatin-based neoadjuvant chemotherapy or nephroureterectomy (RNU)) when a cancer is detected. In one aspect, the gene alteration comprises a TP53, TERT, MYC, FGFR3, CDKN2A, ATM, or ARIDIA alteration.

Also disclosed herein are methods of treating, inhibiting, reducing, decreasing, ameliorating, and/or preventing a cancer and/or metastasis of any preceding aspect, further comprising extracting DNA from the tissue sample.

In one aspect, disclosed herein are methods of treating, inhibiting, reducing, decreasing, ameliorating, and/or preventing a cancer and/or metastasis of any preceding aspect, further comprising measuring plasma copy number burden (CNB); wherein a CNB of >6.5 indicates the presence of a cancer.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Methods

Patients with high-grade cTa-T2 UTUC without radiographic evidence of metastatic disease undergoing up-front radical nephroureterectomy (RNU) were prospectively accrued.

Blood was collected preoperatively on the day of surgery, and plasma and buffy coat were processed for extraction of ctDNA. FFPE samples from RNU were used for tissue genomic DNA extraction. Next-generation sequencing (NGS) was used for variant profiling.

Detection of cancer variants with a mutation allele frequency (MAF)≥0.25% and hotspot variants with a MAF down to 0.1% were reported for plasma samples targeted by a NGS panel (PredicineCARE™). Variants with MAF≥5% and hotspot variants with a MAF down to 2% were reported for FFPE samples.

### b) Results

NGS successfully detected ctDNA in all 15 accrued UTUC patients.

Urothelial tumor tissue: TERT promoter (62%), TP53 (38%), FGFR3 (31%), ERBB2 (25%), ARID1A (19%), and PIK3CA (19%)

Plasma ctDNA: TERT promoter (47%), TP53 (30%), ARIDIA (20%), ERBB2 (20%), FGFR3 (20%), and PIK3CA (17%).

Five patients (33%) had detectable plasma ctDNA mutations concordant with tumor-based genotypes using the targeted NGS panel.

TABLE 1
Baseline clinicopathologic information
and surgical pathology (N = 15)

| | |
|---|---|
| Age, mean (range) | 74 (42-87) |
| Caucasian, N (%) | 15 (100%) |
| Male | 11 (73%) |
| Location | |
| Renal pelvis | 10 (67%) |
| Ureter | 5 (33%) |
| Laterality | |
| Left | 10 (67%) |
| Right | 5 (33%) |
| Pathologic Grading | |
| Low Grade | 3 (20%) |
| High Grade | 12 (80%) |
| Multifocality | |
| Unifocal | 10 (67%) |
| Multifocal | 5 (33%) |
| T Stage | |
| Ta, T1, Tis | 9 (60%) |
| ≥T2 | 6 (40%) |
| N Stage | |
| N0 or Nx | 12 (80%) |
| N+ | 3 (20) |
| Margins | |
| Negative | 13 (87) |
| Positive | 2 (13) |

All patients with detectable preoperative ctDNA had advanced staging (≥pT2 or ≥pN1) and lymphovascular invasion
→sensitivity 71.4%
No patients with pTis/a/1 and pN0 had detectable concordant ctDNA mutations
→specificity 100%

### c) Discussion

Prospective ctDNA analysis using a targeted NGS panel is a feasible nonsurgical approach to predict high-risk UTUC and has the potential to identify patients that may benefit from NAC.

### 2. Example 2: Novel Use of ctDNA to Identify Muscle-Invasive and Non-Organ Confined Upper Tract Urothelial Carcinoma

Upper tract urothelial carcinoma (UTUC) is an aggressive disease with up to 70% incidence of high-grade histology and 60% muscle-invasive staging at the time of radical nephroureterectomy (RNU). Patients with muscle-invasive UTUC (≥pT2) have a poor prognosis, with 5-year cancer-specific mortality rates ranging between 21-59%. Fortunately, there is emerging evidence that cisplatin-based neoadjuvant chemotherapy (NAC) can be safely delivered to achieve pathologic down-staging and improved survival. However, a major challenge preventing optimal patient selection for NAC rests with the difficulty of accurate clinical staging due to UTUC's cloistered anatomical location. Tumor biopsies by ureteroscopy under-stage UTUC up to 46% of the time. Efforts to improve clinical risk stratification using nomograms incorporating ureteroscopic findings, histologic features and cross-sectional imaging yielded only incremental gains. Moreover, clinical under-staging causes missed opportunities for systemic therapy, as those patients who develop renal insufficiency after surgery can no longer qualify for chemotherapy. Given the high stakes for accurate preoperative risk stratification, predictive biomarkers for invasive UTUC are critically needed.

The detection of circulating tumor DNA (ctDNA), that is, plasma cell-free DNA with tumor-specific alterations, is increasingly adopted for numerous clinical applications including cancer diagnosis, assessment of treatment response, and detection of residual disease and/or recurrence ctDNA can be detected in up to 35% of patients with localized urothelial carcinoma of the bladder and 83% with metastatic urothelial cancer. Saliently, higher levels of ctDNA have been shown to correlate with disease burden and portend worse outcomes. It was demonstrated higher levels of ctDNA in patients with muscle invasive bladder cancer than those with recurrent non-muscle invasive disease. Based on these findings, we hypothesized that the detection of plasma ctDNA can be used to refine clinical staging in high-risk UTUC patients undergoing extirpative surgery. In this study, we demonstrate the feasibility of preoperative ctDNA collection and correlate its accuracy in the prediction of muscle-invasive and non-organ confined UTUC (MI/NOC UTUC).

### a) Patients and Methods

### (1) Study Design, Patient Selection, and Clinical Sample Collection

Following IRB approval, 32 patients diagnosed with clinically respectable, high-risk UTUC and planning up-front RNU or ureterectomy at H. Lee Moffitt Cancer Center were prospectively enrolled from October 2020 to April 2022. All patients provided written informed consent and the study was approved by the institutional review board. Treatment and surveillance were performed in accordance with the NCCN and EAU guidelines on UTUC. Recommendations to forego neoadjuvant chemotherapy, the performance of template-based lymphadenectomy, and administration of adjuvant treatment were made by the multidisciplinary treatment team. Pathologic specimens were reviewed by board-certified genitourinary pathology specialists and classified according to the AJCC Cancer Staging Manual, 8th edition. Postsurgical surveillance consisted of cross-sectional imaging, cystoscopy, and urine cytology every 3-6 mo in the first two years and every 6-12 mo thereafter according to the pathologic stage and grade.

Peripheral blood (10 ml) was collected in EDTA-containing tubes (Streck cell-free DNA BCT, La Vista, Nebraska, USA) 1-2 hours prior to surgery. Two-step centrifugation of whole blood was performed at 1600×g for 10 minutes followed by 3200×g for 10 minutes at 10° C. Plasma, buffy coat, and cell pellet were stored at −80° C. From the nephroureterectomy specimen, thick 20 μm sections of formalin-fixed paraffin embedded (FFPE) tumor tissue with ≥50% tumor cellularity were microdissected for
nucleic acid extraction.

### (2) DNA Extraction, Next-Generation Sequencing, and Bioinformatics Analysis

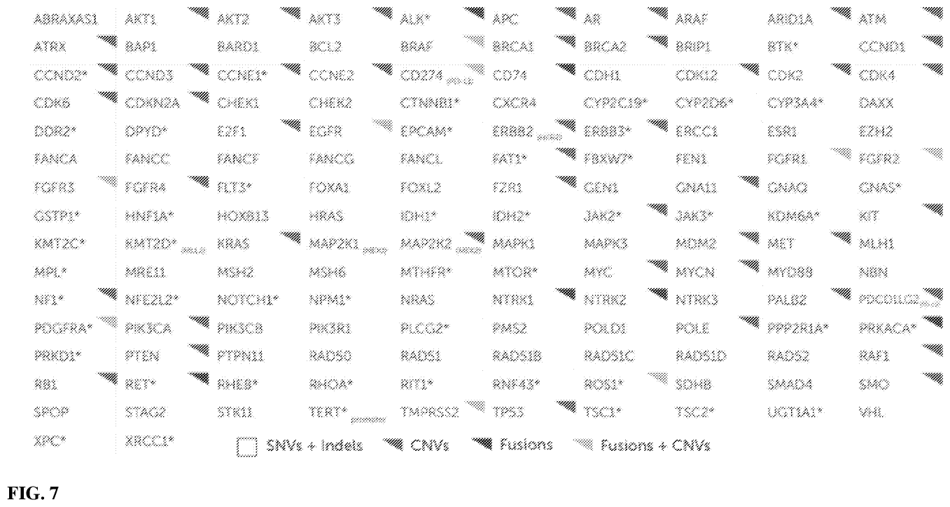

Peripheral blood mononuclear cell (PBMC)-derived germline DNA (gDNA), tumor tissue DNA, and plasma cell-free DNA (cfDNA) were extracted using a combination of established proprietary kits and in-house column-based methods. Thirty of 32 patients had adequate preoperative plasma samples for ctDNA extraction. After quality assessment and quantification, up to 250 ng of gDNA, 50-100 ng of tumor DNA, and 5-30 ng of cfDNA were used for next-generation sequencing library preparation, panel-based hybridization (152-gene PredicineCARE® panel) (FIG. 7), and enrichment prior to ˜20,000X, 150 bp paired-end sequencing on the Illumina NovaSeq 6000 platform (Illumina, San Diego, CA). In parallel, plasma samples were also sequenced using low-pass (1-3×) whole-genome sequencing (WGS).

A proprietary machine learning bioinformatics pipeline (Predicine DeepSEA®) was used to identify single nucleotide variations (SNVs), insertions/deletions (indels), gene-level copy number changes (CNAs), and targeted gene fusions. This algorithm incorporates customized probabilistic control for sequencing errors, detects and eliminates mutations potentially resulting from clonal hematopoiesis of indeterminate potential (CHIP), and calls mutations passing validated allele frequency thresholds of 0.25% (or 0.1% for hotspot mutations) in plasma and 5% (or 2% for hotspot mutations) in FFPE. Additionally, germline mutations detected in matched PBMCs or at high frequency in population genomic databases were filtered out. Pathogenicity of mutations was annotated according to the Clinvar data base. Tumor mutation burden (TMB) was calculated using high-confidence mutations and adjusted for the panel size. In parallel, the low-pass WGS data from plasma cfDNA was used to conduct chromosomal-level copy number analysis using a modified version of the ichorCNA algorithm. Genome-wide copy number burden (CNB) was calculated as an aggregate score across significant copy number changes detected in 1 MB windows throughout the autosomal genome. Using low-pass WGS data, we tested for evidence of somatic chromosomal arm copy number changes in the plasma cfDNA.

### (3) Outcomes and Statistical Analyses

The primary objective was to investigate the ability of plasma ctDNA to distinguish between muscle-invasive/non-organ confined (MI/NOC) and non-muscle invasive (NMI) UTUC. The predictive performance of ctDNA for preoperative identification of MI/NOC UTUC was summarized across preoperative variant count thresholds by calculating the area under the receiver-operating characteristic curve (AUC). The optimal variant count threshold for best sensitivity and specificity was determined using Youden's J statistic implemented in the R package pROC. Based on this method, preoperative ctDNA positivity was defined as the detection of at least two plasma variants, coinciding with other published analyses. The Kaplan-Meier method was used to estimate survival and Mantel-Cox log-rank testing to assess associations between preoperative ctDNA positivity and clinical outcomes including 1) overall survival (OS, time from surgery until UC-related death) and 2) progression-free survival (PFS, time from surgery until progression to metastatic UC). Differences between NMI and MI/NOC patient groups were tested using the Wilcoxon test for continuous variables and the Fisher Exact test for categorical variables. Univariate analysis of each variable was done using logistic regression, and elastic-net regularization was imposed for multivariate models (R packages glm, caret and glmnet). All tests were conducted in R version 4.1.3.

### (4) cfDNA and Genomic DNA Extraction

Whole blood samples were shipped to Predicine on ice and later centrifuged in the lab. Circulating cell-free DNA was extracted from the plasma fraction by QIAamp circulating nucleic acid kit. Quantity and quality of the purified cfDNA was checked using Qubit fluorimeter and Bioanalyzer 2100. For samples with severe genomic contamination from peripheral blood cells, a bead-based size selection was performed to remove large genomic fragments. Genomic DNA (gDNA) was also extracted from the matched buffy coat fraction (PBMCs) from each blood sample and matched patient FFPE tumor sample. Up to 250 ng genomic DNA from PBMC and up to 100 ng gDNA was then enzymatically fragmented and purified.

### (5) Library Preparation, Capture and Sequencing

Five to thirty nanograms of extracted cfDNA were used for plasma cfDNA library construction including end-repair, dA-tailing, and ligation of unique molecular identifiers (UMIs) and sequencing adapters. Ligated fragments were amplified via PCR. The amplified DNA libraries were then further checked using Bioanalyzer 2100 and samples with sufficient yield were used for hybrid capture. When available, a subsample of material was reserved for later low-pass WGS.

Library capture was conducted using Biotin labeled DNA probes. In brief, the library was hybridized overnight with the PredicineCARE panel and paramagnetic beads. The unbound fragments were washed away, and the enriched fragments were amplified via PCR. The purified product was checked using a Bioanalyzer 2100 and then loaded onto the Illumina NovaSeq 6000 for NGS sequencing with paired-end 2×150 bp sequencing kits.

### (6) Analyses of NGS Data

Data was analyzed using the Predicine DeepSea analysis pipeline, which starts from the raw sequencing data (BCL files) and outputs final variant calls. Briefly, the pipeline first does adapter trim, barcode checking, and correction followed by paired FASTQ read alignment to human reference genome build hg19 using the BWA. Candidate variants, consisting of point mutations, small insertions and deletions, are identified across the targeted regions covered in the panel.

### (7) Variant Calling and Annotation

Candidate variants with low base quality, mapping scores, and other quality metrics are filtered. Sequencing and PCR errors are also corrected using the algorithm described in (Newman et al. 2016). In general, a variant identified in cfDNA was considered a somatic mutation only when (i) at least three distinct fragments (at least one of them double-stranded) contained the mutation; and (ii) the mutant allele frequency was higher than 0.25%, or 0.1% for hotspot mutations; and (iii) the ctDNA variant containing fragments are significantly over-represented in comparison with the matched PBMC sample using a fisher-exact test (p-value<0.01 and odds-ratio>3). Non-hotspot variants with high variant frequency (>30%) were considered as suspicious germline variants. For FFPE samples we included variants with >5% MAF, >2% MAF in hotspots.

Candidate somatic mutations were annotated for their effect on protein coding genes as well as probable pathogenicity using the Clin Var database and annotation tool Varsome [ref]. Intronic and silent changes were excluded from our analyses, while missense mutations, nonsense mutations, frameshifts, or splice site alterations were retained. We also excluded common germline variants annotated in the 1000 genomes, ExAC, gnomAD and KAVIAR databases with population allele frequency>0.5%. Finally, we excluded hematopoietic expansion (CHIP) related variants, including those in DNMT3A, ASXL1, TET2.

### (8) Copy Number Alterations and Copy Number Burden

Copy number variation was first estimated at the gene level using the NGS panel data. The in-house pipeline calculates the on-target unique fragment coverage based on consensus bam files, which are first corrected for GC bias and then adjusted for probe-level bias (estimated from a pooled reference). Each adjusted coverage profile is self-normalized (assuming diploid status of each sample) and then compared against correspondingly adjusted coverages from a group of normal reference samples to estimate the significance of each copy number variant. To call an amplification or deletion of a gene, we required the absolute z-score and copy number change to pass minimum thresholds.

We measured genome-wide copy-number burden with PredicineCNB™ (ref). The ichorCNA algorithm [Adalsteinsson V A, et al.] was applied to GC and mappability-normalized reads to estimate plasma and tissue copy number variations using a hidden Markov model (HMM). Firstly, we measured segment level (1 MB) copy number deviation as the log 2 ratio of the normalized reads between a sample and normal plasma background (or used a normal gDNA background for tissue CNB), then we quantified arm-level CNV deviation as the average of segment CNVs across each chromosome arm. Our method also takes into account local cfDNA fragment-size distributions. Finally, we calculated sample-level copy number burden (CNB score) as the sum of absolute zscore of arm-level CNV deviation, where higher CNB score indicates greater absolute CNV abnormality compared with normal background.

### (9) Gene Fusions

DNA rearrangement was detected by identifying the alignment break points based on the BAM files before consensus filtering. Suspicious alignments were filtered based on repeat regions, local entropy calculation and similarity between reference and alternative alignments. Larger than 3 unique alignments (at least one of them double stranded) were required to report a DNA fusion.

### (10) Tumor Fraction

ctDNA fraction was estimated based on the mutant allele fraction of autosomal somatic mutations. Briefly, under the conservative assumption that each SNV may have loss of heterozygosity, the mutant allele fraction (MAF) and ctDNA fraction are related as MAF=(ctDNA*1)/[(1−ctDNA)*2+ctDNA*1], and so ctDNA=2/((1/MAF)+1). Somatic mutations in genes with a detectable copy number change were omitted from ctDNA fraction estimation, thus only a subset of samples could have ctDNA fraction accurately estimated from mutation data.

### (11) bTMB Score Estimation

Blood-based tumor mutational burden (bTMB) was defined as the number of somatic coding SNVs, including synonymous and nonsynonymous variants, within panel target regions. Because TMB estimation considers all variants (including synonymous and non-whitelist variants), higher variant call specificity is required. More stringent cut-offs were used for variant calls, and only variants with allele frequency≥0.35% were used in score calculation. The bTMB score was weighted and normalized by the total effective targeted panel size within the coding region. 43 samples with the maximum somatic allelic frequency (MSAF)<0.7% were excluded for bTMB estimation.

### b) Results

### (1) Patient Characteristics and UTUC Staging

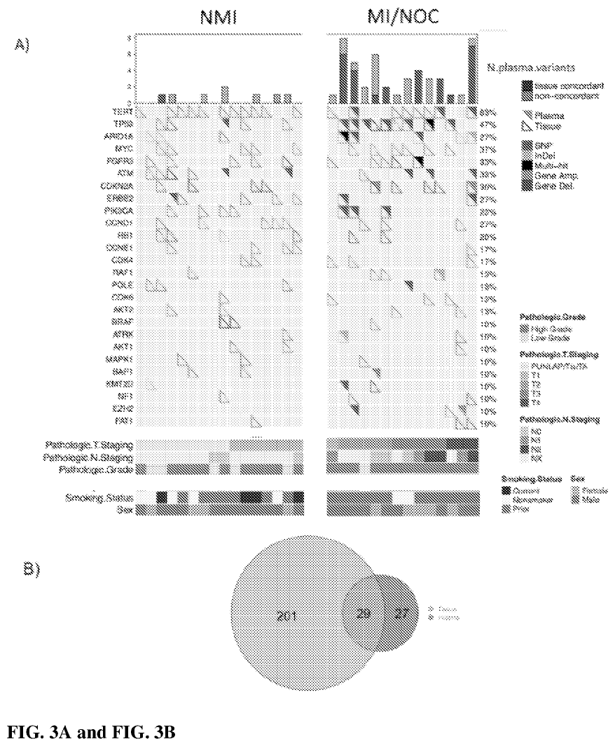

Overall, 30/32 patients with clinically high-risk UTUC undergoing surgical extirpation had preoperative plasma ctDNA passing quality control (QC). Of the two samples failing QC, one was due to processing error and another due to insufficient DNA yield. Of the remaining 30 patients, median age was 74 years (IQR 67, 77.8) and 21 (70%) were male (Table 2). The tumor was located in the renal pelvis in 10 (33%), the ureter in 9 (30%), and both in 11 (37%). Twenty-seven (90%) patients underwent nephroureterectomy, 3 (10%) segmental ureterectomy, and 12 (40%) concomitant regional lymphadenectomy. Two (7%) patients received topical therapy before definitive surgical extirpation and 6 (20%) received adjuvant/salvage chemotherapy. On surgical pathology, 24 (80%) were high grade and 13 (43%) were MI UTUC (≥pT2). Six (20%) patients were found to harbor nodal metastases. Interestingly, one patient with pT1 HG ureteral tumor was found to have occult nodal metastasis, resulting in 14/30 (47%) patients with MI/NOC UTUC (FIG. 3).

TABLE 2
Clinicopathologic and treatment characteristics (N = 30)

| | |
|---|---|
| N | % |

| Age, median | 74 | years | range 67-77.8 years |
|---|---|---|---|
| Race | | | |
| Caucasian | 28 | | 93 |
| Hispanic | 1 | | 3 |
| Asian | 1 | | 3 |
| Gender | | | |
| Male | 21 | | 70 |
| Female | 9 | | 30 |
| Smoking Status | | | |
| Current smoker | 4 | | 13 |
| Prior smoker | 19 | | 63 |
| Nonsmoker | 7 | | 23 |
| History of bladder cancer | 13 | | 43 |
| Prior radical cystectomy | 3 | | 10 |
| Laterality | | | |
| Left | 21 | | 70 |
| Right | 9 | | 30 |
| Preoperative | 13 | | 43 |
| hydronephrosis Tumor | | | |
| location | | | |
| Renal pelvis | 10 | | 33 |
| Ureter | 9 | | 30 |
| Both | 11 | | 37 |
| Tumor size, median | 3.8 | cm | 2.6, 5.6 IQR |
| <2 cm | 5 | | 17 |
| >2 cm | 21 | | 70 |
| Multifocal | 13 | | 43 |
| Variant histology | 2 | | 7 |
| Pathologic grading | | | |
| Low-grade | 6 | | 20 |
| High-grade | 24 | | 80 |
| Pathologic tumor stage | | | |
| Ta, Tis, T1 | 17 | | 56 |
| T2 | 5 | | 17 |
| T3 | 5 | | 17 |
| T4 | 3 | | 10 |
| Pathologic node stage | | | |
| Nx | 18 | | 60 |
| N0 | 6 | | 20 |
| N1 | 3 | | 10 |
| N2 | 3 | | 10 |
| Follow up, median | 12.7 | months | 8.5, 17.5 IQR |

| | | |
|---|---|---|
| Urothelial recurrence | 9 | 30 |
| Metastatic Progression | 6 | 20 |
| Death | 5 | 17 |

### (2) Somatic Mutations and CNAs

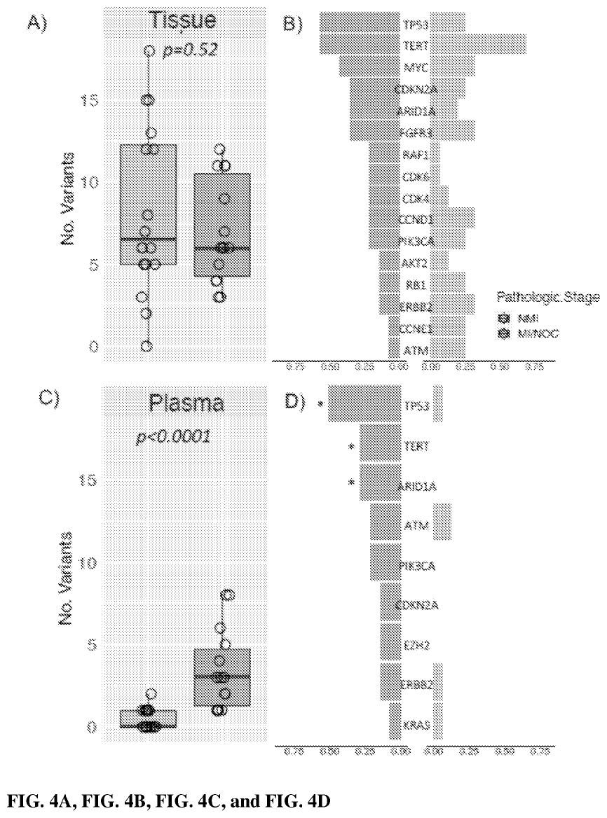

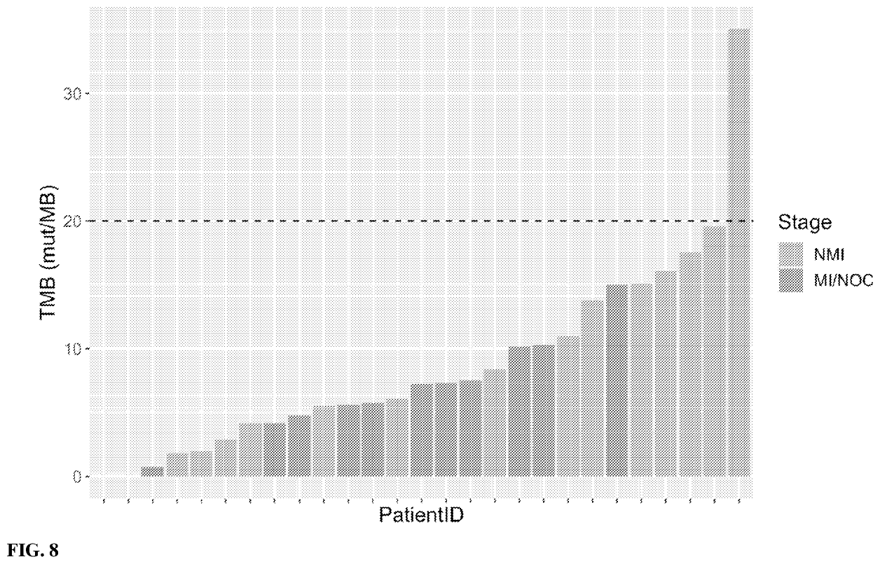

The 152-gene PredicineCARE™ panel covers 81.2% of the commonly altered genes (≥10% incidence) in UTUC. To investigate the concordance between plasma and tumor tissue-derived DNA, targeted sequencing was performed on matching plasma and surgical UTUC samples. Overall, molecular alterations (MA) including SNVs/indels (66%) and gene-level CNAs (34%) were detected in 29 out of 30 (97%) tumor samples, spanning 75 of the 152 paneled-genes (FIG. 3a). In addition, 1 FGFR3-TACC3 fusion was found. Of the SNVs and indels, 29% were classified as Pathogenic. Each tumor contained a median of 6 (range 0-18) MAs and a mean TMB of 8.8 mutations/Mb (range 0-35.1), similar to levels (FIG. 8). One (3.3%) hypermutated tumor was found within our cohort, consistent with the 5.5% incidence described by others. Interestingly, this patient did not have germline mismatch repair gene alterations or prior cancer history. The most common tumor derived variants included TERT promoter (63%), TP53 (40%), MYC (37%), FGFR3 (33%), and CDKN2A (30%) (FIG. 4b). Although there was no significant difference in the total number of variants detected between NMI vs MI/NOC tumor tissue (8.3 vs 7, p=0.5, FIG. 4a), important distinctions exist at the gene level. TP53 (57% vs. 25%, p=0.1) was more commonly mutated in MI/NOC UTUC though this difference did not reach statistical significance. In contrast, a similar prevalence of FGFR3 (31% vs. 31%) and TERT promoter (69% vs. 57%) alterations were found in NMI and MI/NOC tumors (FIG. 4b).

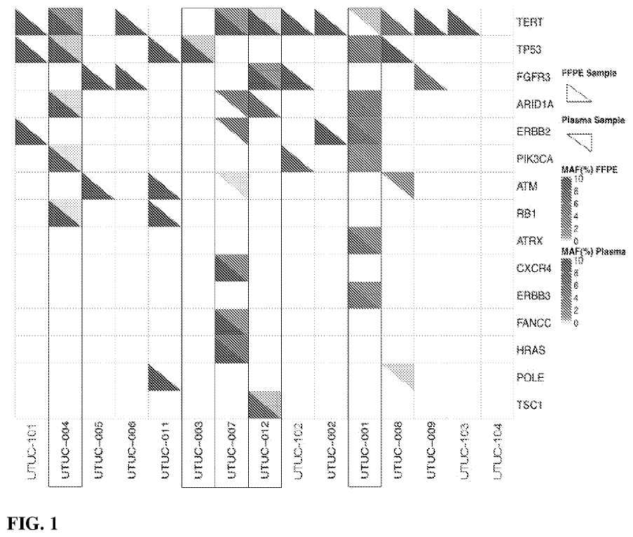

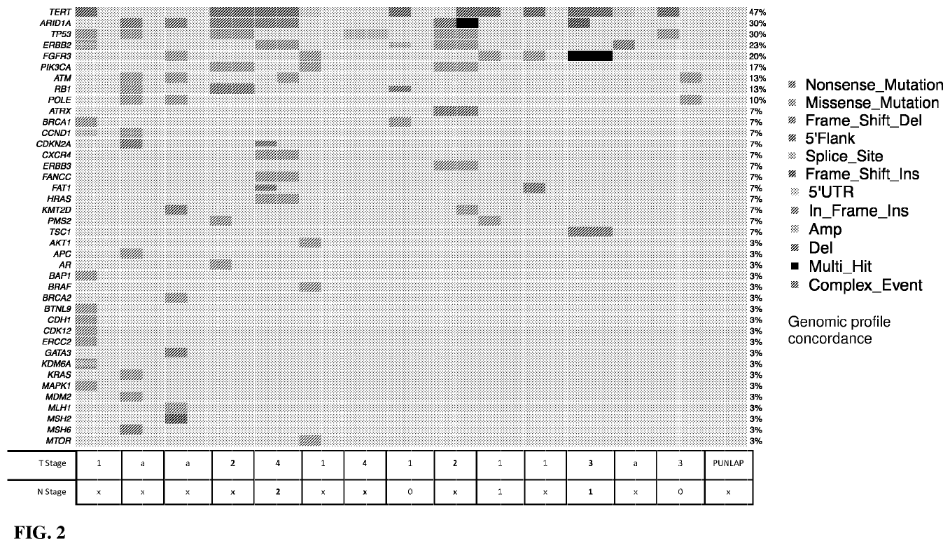

At least one MA was detected within the cfDNA from 21/30 (70%) preoperative plasma samples, with each patient carrying a median of 1 ctDNA variant (range 0-8) (FIG. 3a). The most commonly detected alterations in the plasma cfDNA were TP53 (27%), ATM (17%), and ARIDIA (13%) (FIG. 4d). Overall, 52% of the detected plasma ctDNA variants corroborated alterations detected in the matching tumor samples. On the other hand, 88% of the tumor variants were not detected within the plasma (FIG. 1b). In particular, alterations in TP53, ARIDIA, and PIK3CA were frequently detected concomitantly in paired plasma and tissue samples. Importantly, a significantly higher number of plasma variants were detected within the plasma from MI/NOC (mean 3.4, range 1-8) vs. NMI UTUC (mean 0.5, range 0-2, p<0.0001) (FIG. 4c).

### (3) Clinical Utility of Preoperative ctDNA Detection and Staging

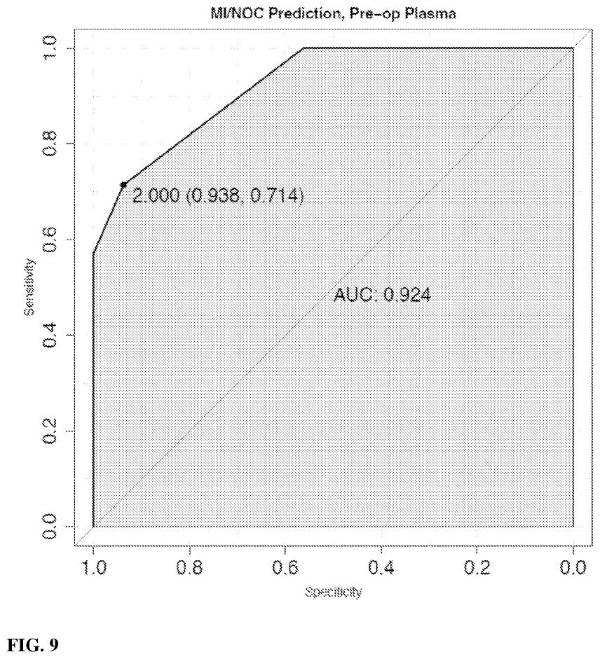

The detection of ctDNA has been utilized in the preoperative setting to estimate tumor burden and refine prognosis in patients with stage III cutaneous melanoma. Similarly, we evaluated the utility of preoperative ctDNA to predict clinical staging in our cohort. Following summarization of predictive performance for optimal sensitivity and specificity of preoperative identification of MI/NOC UTUC across variant count thresholds, the presence of pre-surgical ctDNA was defined as the detection of at least two panel-based plasma MAs. Detection of ctDNA at this threshold was strongly predictive of MI/NOC UTUC at the time of surgery, achieving a sensitivity of 71% and specificity of 94% for detecting MI/NOC UTUC stage with an AUC of 0.92 (0.85-1.0, 95% CI) (FIG. 9). From 14 MI/NOC patients, four false negatives occurred, in patients with HG-UTUC staged pT1N1, pT3Nx, pT4Nx, and pT4N1. The one false positive occurred in a patient with multifocal HG pTaNO with a large 6.1 cm renal pelvic tumor.

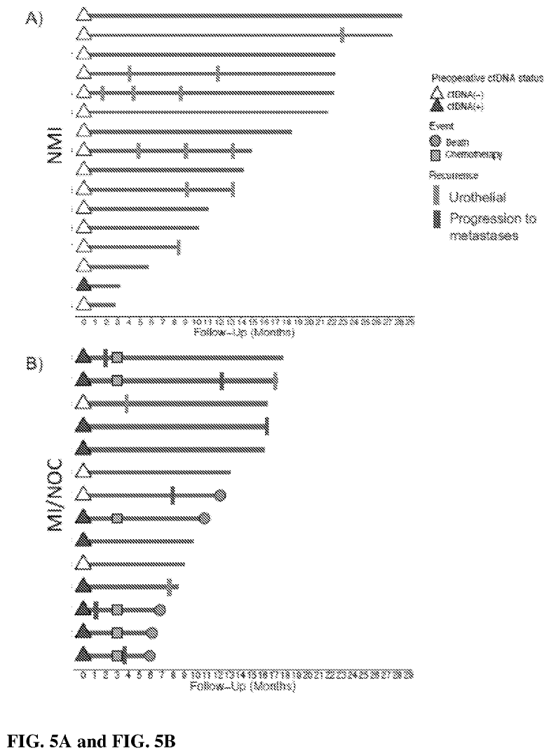

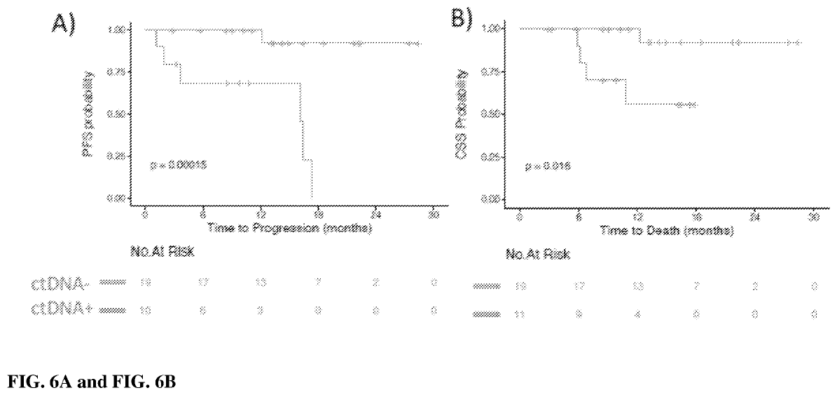

Moreover, the presence of ctDNA preoperatively was strongly prognostic. At a median follow-up of 12.7 months (Range 8.5-17.5 months), 6 (20%) patients suffered progression to metastases and 5 (17%) disease-specific death. Of the 11 patients with detectable ctDNA, 5 (45%) were found to have disease progression, 3 (27%) died of UTUC, and 1 (9%) died of unknown causes (FIG. 5). In contrast, 1 of 19 (5%) patients with negative ctDNA suffered disease progression and died (FIG. 5). Patients with detectable preoperative ctDNA had significantly shorter progression-free (one-year PFS 69% vs 100%, p<0.001) (FIG. 6a) and overall survival (56% vs 100%, p<0.02) (FIG. 6b). ctDNA positivity predicted both shorter PFS (HR=20.4, 95% CI [2.4, 174.3], p<0.0001) and OS (HR=9.3, 95% CI [1-84], p=0.02). In contrast, preoperative ctDNA positivity was not significantly associated with urothelial recurrence-free survival (one-year RFS 57% vs 59%, p=0.1).

### (4) Utility of Copy Number Changes and Staging

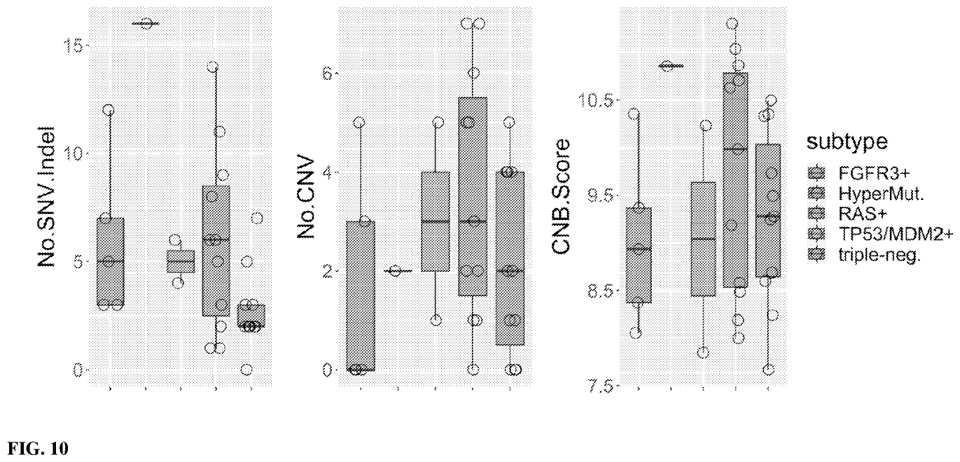

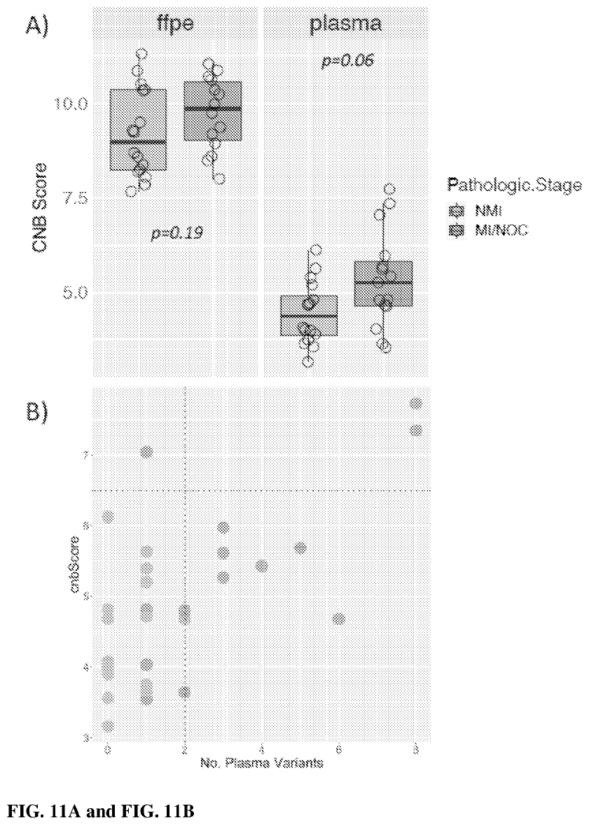

There is a dichotomy between UTUCs with highly complex karyotypes containing frequent focal CNAs, aneuploidy, and chromothripsis compared to those with simple arm-level aberrations most commonly involving chromosome 1q, 3, 8, and 9. Furthermore, those with complex CNAs were frequently found to have TP53 alterations, and more likely to be staged as muscle-invasive and exhibit aggressive phenotypes. We hypothesized that high CNB correlates with pathologic staging and can be used to complement ctDNA positivity to improve the prediction model for MI/NOC UTUC. Evidence of somatic chromosomal arm gain and/or loss was found in all tumors. TP53-altered tumors had numerically higher number of CNAs than other subtypes (3.5 vs. 2.1, p=0.09) (FIG. 10). Overall, there was a marginal difference in preoperative plasma CNB score (4.5 vs. 5.2, p=0.06) between NMI and MI/NOC UTUC patients (FIG. 11A). Imposing a threshold of plasma CNB score>6.5 to confirm MI/NOC UTUC when at least two plasma variants were detected increased the sensitivity of prediction to 79%, without compromising the specificity (FIG. 11B).

### c) Discussion

The inability to accurately stage tumors prior to surgical extirpation has severely hampered customization of treatment for patients with high risk UTUC. As evidenced herein, despite adherence to clinical guidelines, 30% of the patients suffered rapid disease progression and/or cancer-specific death within two years following surgery with curative intent. With emerging evidence supporting the use of neoadjuvant chemotherapy, clinical tools to enhance the identification of potential beneficiaries with invasive, micrometastatic disease prior to surgery are critically needed. The inherent loss of renal function from surgical extirpation renders a subset of patients ineligible for adjuvant cisplatin-based chemotherapy, which makes this unmet need even more dire. Against this backdrop, our study provides encouraging evidence that plasma ctDNA collected at diagnosis estimates tumor burden and can be leveraged to distinguish between patients with MI/NOC from those with NMI UTUC. Equally important, ctDNA was strongly prognostic for disease progression and death following surgery, making it a promising biomarker for selecting patients to undergo chemotherapy in the neoadjuvant setting.

When treating high risk UTUC, more emphasis should be placed on minimizing missed opportunities to provide life-prolonging systemic treatment to patients with MI/NOC UTUC undergoing extirpative surgery. To that end, the sensitivity of preoperative ctDNA to define MI/NOC UTUC reached 79% in our model based on the detection of at least 2 panel-based plasma variants and a minimal threshold of plasma CNB score of 6.5. Although needing validation, this level of sensitivity represents a clear improvement over the more modest sensitivities between 42-48% achieved using available clinical nomograms.

On top of its clinical relevance, the 152-gene PredicineCARE™ ctDNA platform provided ease of clinical application and high genomic fidelity. Of the 31 plasma samples appropriately processed, only 1 failed to yield sufficient cfDNA for analysis. From the analysis of tumor tissue samples, MAs were detected in all but one of the samples (97%), validating the broad coverage of the frequently altered genes in UTUC.

Due to the rare incidence of UTUC, scant data exists on the application of ctDNA in the localized UTUC setting. Using a 73-gene panel, ctDNA were defined as one or more tumor-derived MAs and reported detection of ctDNA in 95% of 75 metastatic UTUC patients from 13 academic institutions, with an average of 6.8 Mas per patient. The higher plasma MA rates can reflect higher disease burden in patients with metastatic disease, though sporadic UTUC has also been shown to have a lower mutational burden than urothelial carcinoma of the bladder. Similar to our study, the most frequently encountered plasma MAs were TP53 (51%), PIK3CA (23%), ARIDIA (20%), and TERT (17%), albeit at higher detection frequencies. Likewise, frequent chromosomal arm-level gains and losses in addition to focal CNAs at the 9p24.3 region (CD274, JAK2, and PDCD1LG2) and 1q21.3-1q23.3 (PVRL4) were detected in our study. These genome-wide measures of plasma cfDNA CNB were also associated with advanced staging (5.2 vs. 4.5, p=0.06). Adding CNB as a secondary predictor of MI/NOC UTUC to our model increased the sensitivity from 71% to 79% (FIG. 11B). Our study represents the first investigation at scale of the ctDNA landscape in localized, high-risk UTUC and its concordance with somatic mutations found from matched tumor specimens.

### d) Conclusion

117. In this prospective observational study, we demonstrate the clinical utility of ctDNA managing high risk, localized UTUC. Preoperative ctDNA positivity based on the detection of at least 2 plasma variants and a minimal threshold of CNB score of 6.5 was highly predictive of MI/NOC UTUC staging and strongly prognostic of progression and overall survival. Preoperative ctDNA analysis is feasible and can be used to select high risk UTUC patients benefiting from neoadjuvant chemotherapy.

### E. REFERENCES

Adalsteinsson V A, Ha G, Freeman S S, Choudhury A D, Stover D G, Parsons H A, et al. Scalable whole-exome sequencing of cell-free DNA reveals high concordance with metastatic tumors. Nat Commun. 2017; 8:1324.
Agarwal N, Pal S K, Hahn A W, Nussenzveig R H, Pond G R, Gupta S V, et al. Characterization of metastatic urothelial carcinoma via comprehensive genomic profiling of circulating tumor DNA. Cancer. 2018; 124:2115-24.
Audenet F, Isharwal S, Cha E K, Donoghue M T A, Drill E N, Ostrovnaya I, et al. Clonal Relatedness and Mutational Differences between Upper Tract and Bladder Urothelial Carcinoma. Clinical Cancer Research. 2019; 25:967-76.
Baard J, de Bruin D M, Zondervan P J, Kamphuis G, de la Rosette J, Laguna M P. Diagnostic dilemmas in patients with upper tract urothelial carcinoma. Nat Rev Urol. 2017; 14:181-91.
Barata P C, Koshkin V S, Funchain P, Sohal D, Pritchard A, Klek S, et al. Next-generation sequencing (NGS) of cell-free circulating tumor DNA and tumor tissue in patients with advanced urothelial cancer: a pilot assessment of concordance. Ann Oncol. 2017; 28:2458-63.
Birkenkamp-Demtröder K, Nordentoft I, Christensen E, Høyer S, Reinert T, Vang S, et al.
Genomic Alterations in Liquid Biopsies from Patients with Bladder Cancer. Eur Urol. 2016; 70:75-82.
Chalfin H J, Glavaris S A, Gorin M A, Kates M R, Fong M H, Dong L, et al. Circulating Tumor Cell and Circulating Tumor DNA Assays Reveal Complementary Information for Patients with Metastatic Urothelial Cancer. Eur Urol Oncol. 2021; 4:310-4.
Christensen E, Birkenkamp-Demtröder K, Sethi H, Shchegrova S, Salari R, Nordentoft I, et al. Early Detection of Metastatic Relapse and Monitoring of Therapeutic Efficacy by Ultra-Deep Sequencing of Plasma Cell-Free DNA in Patients With Urothelial Bladder Carcinoma. J Clin Oncol. 2019; 37:1547-57.
Christensen E, Nordentoft I, Birkenkamp-Demtroder K, Elbæk SK, Lindskrog S V, Taber A, et al. Cell-free urine- and plasma DNA mutational analysis predicts neoadjuvant chemotherapy response and outcome in patients with muscle invasive bladder cancer. Clin Cancer Res. 2023.
Coleman J A, Yip W, Wong N C, Sjoberg D D, Bochner B H, Dalbagni G, et al. Multicenter Phase II Clinical Trial of Gemcitabine and Cisplatin as Neoadjuvant Chemotherapy for Patients With High-Grade Upper Tract Urothelial Carcinoma. Journal of Clinical Oncology.0: JCO.22.00763.
Corcoran R B, Chabner B A. Application of Cell-free DNA Analysis to Cancer Treatment. N Engl J Med. 2018; 379:1754-65.
Edge S. AJCC cancer staging manual. Springer. 2010; 7:97-100.
Favaretto R L, Shariat S F, Savage C, Godoy G, Chade D C, Kaag M, et al. Combining imaging and ureteroscopy variables in a preoperative multivariable model for prediction of muscle-invasive and non-organ confined disease in patients with upper tract urothelial carcinoma. BJU Int. 2012; 109:77-82.
Fettke H, Kwan E M, Docanto M M, Bukczynska P, Ng N, Graham L-J K, et al. Combined Cell-free DNA and RNA Profiling of the Androgen Receptor: Clinical Utility of a Novel Multianalyte Liquid Biopsy Assay for Metastatic Prostate Cancer. European Urology. 2020; 78:173-80.
Flaig T W, Spiess P E, Agarwal N, Bangs R, Boorjian S A, Buyyounouski M K, et al. Bladder Cancer, Version 3.2020, NCCN Clinical Practice Guidelines in Oncology. J Natl Compr Canc Netw. 2020; 18:329-54.
Fujii Y, Sato Y, Suzuki H, Kakiuchi N, Yoshizato T, Lenis A T, et al. Molecular classification and diagnostics of upper urinary tract urothelial carcinoma. Cancer cell. 2021; 39:793-809.e8.
Green E A, Li R, Albiges L, Choueiri T K, Freedman M, Pal S, et al. Clinical Utility of Cell-free and Circulating Tumor DNA in Kidney and Bladder Cancer: A Critical Review of Current Literature. Eur Urol Oncol. 2021.
Guo G, Sun X, Chen C, Wu S, Huang P, Li Z, et al. Whole-genome and whole-exome sequencing of bladder cancer identifies frequent alterations in genes involved in sister chromatid cohesion and segregation. Nature Genetics. 2013; 45:1459-63.
Kaag M G, O'Malley R L, O'Malley P, Godoy G, Chen M, Smaldone M C, et al. Changes in renal function following nephroureterectomy may affect the use of perioperative chemotherapy. Eur Urol. 2010; 58:581-7.
Landrum M J, Lee J M, Riley G R, Jang W, Rubinstein W S, Church D M, et al. Clin Var: public archive of relationships among sequence variation and human phenotype. Nucleic Acids Res. 2014; 42: D980-5.
Lee J H, Saw R P, Thompson J F, Lo S, Spillane A J, Shannon K F, et al. Pre-operative ctDNA predicts survival in high-risk stage III cutaneous melanoma patients. Annals of Oncology. 2019; 30:815-22.
Margulis V, Puligandla M, Trabulsi E J, Plimack E R, Kessler E R, Matin S F, et al. Phase II Trial of Neoadjuvant Systemic Chemotherapy Followed by Extirpative Surgery in Patients with High Grade Upper Tract Urothelial Carcinoma. Journal of Urology. 2020; 203:690-8.
Margulis V, Youssef R F, Karakiewicz P I, Lotan Y, Wood C G, Zigeuner R, et al. Preoperative multivariable prognostic model for prediction of nonorgan confined urothelial carcinoma of the upper urinary tract. J Urol. 2010; 184:453-8.
Petros F G, Qiao W, Singla N, Clinton T N, Robyak H, Raman J D, et al. Preoperative multiplex nomogram for prediction of high-risk nonorgan-confined upper-tract urothelial carcinoma. Urol Oncol. 2019; 37:292 e1-e9.
Robin X, Turck N, Hainard A, Tiberti N, Lisacek F, Sanchez J C, et al. pROC: an open-source package for R and S+ to analyze and compare ROC curves. BMC Bioinformatics. 2011; 12:77.
Robinson B D, Vlachostergios P J, Bhinder B, Liu W, Li K, Moss T J, et al. Upper tract urothelial carcinoma has a luminal-papillary T-cell depleted contexture and activated FGFR3 signaling. Nat Commun. 2019; 10:2977.
Rose K M H H, Meeks J, et al. Circulating and urinary tumour DNA in urothelial carcinoma-upper tract, lower tract, and metastatic disease. Nature Reviews Urology. 2023; Ahead of print.
Roupret M, Babjuk M, Burger M, Capoun O, Cohen D, Comperat E M, et al. European Association of Urology Guidelines on Upper Urinary Tract Urothelial Carcinoma: 2022 Update. Eur Urol. 2021; 79:62-79.
Ruvolo C C, Nocera L, Stolzenbach L F, Wenzel M, Cucchiara V, Tian Z, et al. Incidence and survival rates of contemporary patients with invasive upper tract urothelial carcinoma. European urology oncology. 2021; 4:792-801.
Sharma V, Miest T S, Juvet T S, Toussi A, Packiam V, Chamie K, et al. The Impact of Upper Tract Urothelial Carcinoma Diagnostic Modality on Intravesical Recurrence after Radical Nephroureterectomy: A Single Institution Series and Updated Meta-Analysis. J Urol. 2021; 206:558-67.
Shohdy K S, Villamar D M, Cao Y, Trieu J, Price K S, Nagy R, et al. Serial ctDNA analysis predicts clinical progression in patients with advanced urothelial carcinoma. Br J Cancer. 2022; 126:430-9.
Soria F, Shariat S F, Lerner S P, Fritsche H M, Rink M, Kassouf W, et al. Epidemiology, diagnosis, preoperative evaluation and prognostic assessment of upper-tract urothelial carcinoma (UTUC). World J Urol. 2017; 35:379-87.
Subiela J D, Territo A, Mercade A, Balana J, Aumatell J, Calderon J, et al. Diagnostic accuracy of ureteroscopic biopsy in predicting stage and grade at final pathology in upper tract urothelial carcinoma: Systematic review and meta-analysis. Eur J Surg Oncol. 2020; 46:1989-97.
Vandekerkhove G, Lavoie J M, Annala M, Murtha A J, Sundahl N, Walz S, et al. Plasma ctDNA is a tumor tissue surrogate and enables clinical-genomic stratification of metastatic bladder cancer. Nature Communications. 2021; 12.
Yoshida T, Kobayashi T, Kawaura T, Miyake M, Ito K, Okuno H, et al. Development and external validation of a preoperative nomogram for predicting pathological locally advanced disease of clinically localized upper urinary tract carcinoma. Cancer Med. 2020; 9:3733-41.
Zhang R, Zang J, Xie F, Zhang Y, Wang Y, Jing Y, et al. Urinary Molecular Pathology for Patients with Newly Diagnosed Urothelial Bladder Cancer. J Urol. 2021; 206:873-84.

1. A method of detecting the presence of a cancer in a subject comprising
obtaining a tissue sample from the subject; and
assaying circulating tumor DNA (ctDNA) in the tissue sample using next generation sequencing (NGS) or whole genome sequencing (WGS) to detect the presence of alternations in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGTIA1, VHL, XPC, and XRCC1;
wherein the presence of two or more genes indicates the presence of a cancer.

2. The method of claim 1, wherein the cancer comprises a bladder or urinary tract cancer.

3. (canceled)

4. (canceled)

5. The method of claim 1, wherein the gene alteration is a somatic alteration.

6. (canceled)

7. The method of claim 1, wherein the tissue sample comprises a liquid biopsy.

8. (canceled)

9. (canceled)

10. The method of claim 1, further comprising measuring plasma copy number burden (CNB); wherein a CNB of >6.5 indicates the presence of a cancer.

11. The method of claim 1, further comprising administering to the subject an anti-cancer treatment when a cancer is detected.

12. (canceled)

13. A method of predicting survival in a subject treated for a cancer or staging the severity of a cancer in a subject, the method comprising
obtaining a tissue sample from the subject; and
assaying circulating tumor DNA (ctDNA) in the tissue sample using next generation sequencing to detect the presence of alternations in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGT1A1, VHL, XPC, and XRCC1;
wherein the presence of two or more genes indicates an aggressive cancer and low chance survival.

14. The method of claim 13, wherein the survival is progression free survival.

15. The method of claim 13, wherein the cancer comprises a bladder or urinary tract cancer.

16. (canceled)

17. (canceled)

18. The method of claim 13, wherein the gene alteration is a somatic alteration.

19. (canceled)

20. The method of claim 13, wherein the tissue sample comprises a liquid biopsy.

21. (canceled)

22. (canceled)

23. The method of claim 13, further comprising measuring plasma copy number burden (CNB); wherein a CNB of >6.5 indicates the presence of a cancer.

24. The method of claim 13, wherein the method is performed after nephroureterectomy (RNU).

25. The method of claim 13, further comprising administering to the subject an anti-cancer treatment when cancer survival is low or an aggressive cancer is detected.

26.-38. (canceled)

39. A method of treating a cancer in a subject comprising
obtaining a tissue sample from the subject;
assaying circulating tumor DNA (ctDNA) in the tissue sample using next generation sequencing (NGS) or whole genome sequencing (WGS) to detect the presence of alternations in one or more genes selected from the group consisting of ABRAXAS1, AKT1, AKT2, AKT3, ALK, APC, AR, ARAF, ARIDIA, ATM, ATRX, BAP1, BARD1, BCL2, BRAF, BRCA1, BRAC2, BRIP1, BTK, CCND1, CCND2, CCND3, CCNE1, CCNE2, CD274, CD74, CDH1, CDK12, CDK2, CDK4, CDK6, CDKN2A, CHEK1, CHEK2, CTNNB1, CXCR4, CYP2C19, CYP2D6, CYP3A4, DAXX, CCR2, CPYD, E2F1, EGFR, EPCAM, ERBB2, ERBB3, ERCC1, ESR1, EZH2, FANCA, FANCC, FANCF, FANCG, FANCL, FAT1, FBXW7, FEN1, FGFR1, FGFR2, FGFR3, FGFR4, FLT3, FOXA1, FOXL2, FZR1, GEN1, GNA11, GNAQ, GNAS, GSTP1, HNF1A, HOXB13, HRAS, IDH1, IDH2, JAK2, JAK3, KDM6A, KIT, KMT2C, KMT2D, KRAS, MAP2K1, MAP2K2, MAPK1, MAPK3, MDM2, MET, MLH1, MPL, MRE11, MSH2, MSH6, MTHFR, MTOR, MYC, MYCN, MYD88, NBN, NF1, NFE2L2, NOTCH1, NPM1, NRAS, NTRK1, NTRK2, NTRK3, PALB2, PDCDILG2, PDGFRA, PIK3CA, PIK3CB, PIK3R1, PLCG2, PMS2, POLD1, POLE, PPP2R1A, PRKACA, PRKD1, PTEN, PTPN11, RAD50, RAD51, RAD51B, RAD51C, RAD51D, RAD52, RAF1, RB1, RET, RHEB, RHOA, RIT1, RNF43, ROS1, SDHB, SMAD4, SMO, SPOP, STAG2, STK11, TERT, TMPRSS2, TP53, TSC1, TSC2, UGTIA1, VHL, XPC, and XRCC1;
wherein the presence of two or more genes indicates the presence of a cancer; and
administering to the subject an anti-cancer treatment when a cancer is detected.

40. The method of claim 39, wherein the cancer comprises a bladder or urinary tract cancer.

41. (canceled)

42. (canceled)

43. The method of claim 39, wherein the gene alteration is a somatic alteration.

44. (canceled)

45. The method of claim 39, wherein the tissue sample comprises a liquid biopsy.

46. (canceled)

47. (canceled)

48. The method of claim 39, further comprising measuring plasma copy number burden (CNB); wherein a CNB of >6.5 indicates the presence of a cancer.

49. The method of claim 39, wherein the anti-cancer treatment comprises a cisplatin-based neoadjuvant chemotherapy or nephroureterectomy (RNU).
